(19) United States
(12) Patent Application Publication
(10) Pub. No.: US 2006/0155116 A1
(43) Pub. Date: Jul. 13, 2006

(54) Hypoallergenic variants of the parietaria judaica major allergens, uses thereof and compostitions comprising them

(76) Inventors: Maria Assunta Costa, Palermo (IT); Domenico Geraci, Palermo (IT); Paolo Colombo, Palermo (IT); Rosa Passantino, Palermo (IT); Angela Bonura, Palermo (IT)

Correspondence Address: Albert Wai Kit Chan;Law Offices of Albert Wai Kit Chan, World Plaza Suite 604, 141 07 20th Avenue, Whitestone, NY, 11357, US

(21) Appl. No.: 10/557,586

(22) PCT Filed: May 19, 2004
(86) PCT No.: PCT/IT04/00284

§ 371 Date: Mar. 3, 2006

(30) Foreign Application Priority Data
May 20, 2003 (IT) RM2003A000247

(51) Int. Cl. A61K 39/35; A61K 36/00; C07K 14/415; C07H 21/04

(52) U.S. Cl. 530/370; 424/275.1; 435/69.3; 435/252.3; 435/419; 435/468; 536/23.6

(57) ABSTRACT

A multimer protein molecule comprising at least a first amino acid sequence having substantially the sequence of one of the Parietaria judaica major allergens and a second amino acid sequence having substantially the sequence of one of the Parietaria judaica major allergens Par j 1 or Par j 2, and a third sequence of one of the Parietaria judaica major allergens Par j 1 or Par j 2. The molecule is also mutated in the amino acid region corresponding to loop 1, so that is hypoallergenic. The molecule can be utilized for medical use as a hypoallergenic agent.

The present invention concerns hypoallergenic protein molecules derived from the Parietaria judaica major allergens, uses and compositions thereof The allergic reaction, also known as type I hypersensitivity reaction, is caused by an IgE-mediated response to normally harmless environmental antigens present in animal dander, dust mites and pollen. Symptoms include wheals and skin eruptions, rhinoconjunctivitis, conjunctivitis, difficulty in breathing and other more severe conditions such as asthma and anaphylaxis.

Hypersensitivity does not manifest at the first contact with the antigen but rather it appears after an initial phase of sensitization. The process is triggered when the allergen comes into contact with antigen presenting cells (APC) whose principal role is to capture the antigen and digest it into small fragments, which are then presented on the cell membrane surface in association with specific glycoproteins, namely the class II major histocompatibility (MHC) antigens.

Respiratory allergies may be classified as seasonal or perennial, depending on the period of the year in which they appear. In seasonal respiratory allergies, the allergens causing the disorder are present in the pollen of plants that bloom chiefly in the spring. The pollen suspended in the atmosphere is inhaled and reaches the mucous membrane of the respiratory tract where the protective envelope surrounding the pollen is dissolved by the enzymes and moisture present in the mucosal secretion, thus releasing the proteins contained in the envelope.

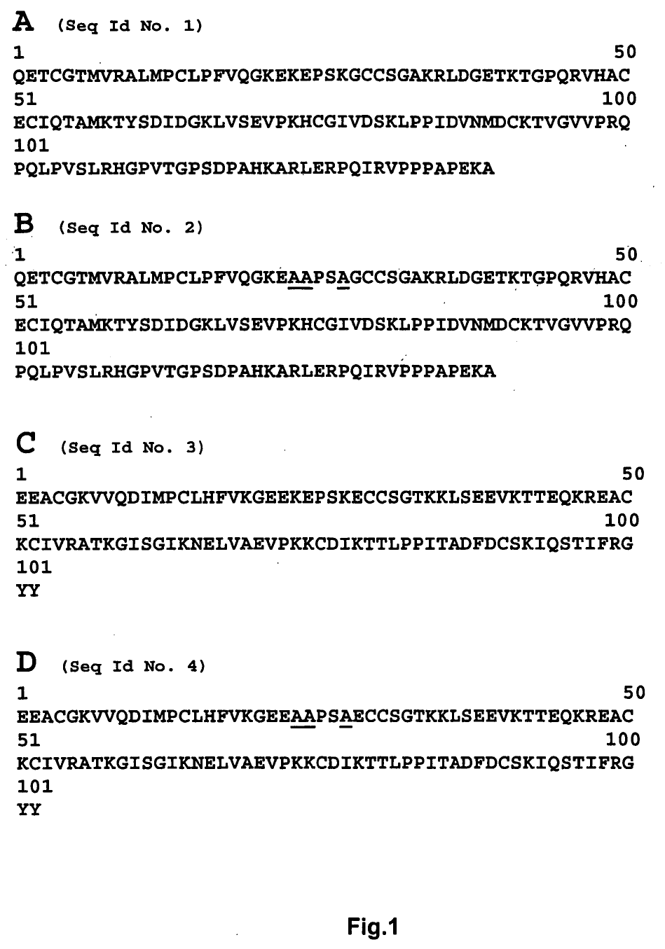

The most common allergenic plant species belong to several large families: Fagaceae, Urticaceae, Oleaceae, composites and Graminaceae. In continental Italy, the Graminaceae family is the main cause of allergic reactions, whereas in the Mediterranean area the main allergenic plant is Parietaria judaica (Pj). The genus Parietaria belongs to the Urticaceae family and includes five species which ranked by allergic importance are. P. judaica, P. officinalis, P. lusitanica, P. cretica and P. mauritanica (1). Early experimental studies using biochemical methods (CIE and CRIE) showed that Pj pollen contains numerous allergens that differ in molecular weight and ability to bind IgE. The range of the molecular weight of the allergens is from 10 to 80 kDa, and those with a weight from 10 to 14 kDa react with all sera of allergic subjects, suggesting that the main allergens can be found in this range (2, 3). Starting from an expression library, the recombinant DNA technique to isolate the Pj major allergens allowed the isolation and characterization of the major allergens, Par j 1 and Par j 2, and several of their isoforms (4). From Par j 1 two variants were isolated, designated Par j 1.0102 and Par j 1.0201. Par j 1.0102 has an insert of 794 nucleotides, a deduced amino acid sequence containing 176 amino acids with a molecular weight of 18.450 Da. The NH₂-terminal sequence has the characteristic amino acid sequence of glycosylated protein signal sequences. The mature protein has 139 amino acids and a molecular weight of 14.476 Da (FIG. 1, panel a). Sequence analysis of the Par j 1.0201 clone showed an insert of 637 nucleotides, an amino acid sequence of 139 amino acids, and a molecular weight of 14.400 Da. It also contains an amino terminal region with signal sequence characteristics. The mature protein contains 102 amino acids and has a molecular weight of 10.677 Da. At the amino acid level, the coding region of Par j 1.0201 is 89% homologous with that of Par j 1.0102, but the lack of homology in the 3′ and 5′ untranslated regions suggests that the two clones derive from the transcription of independent genes. In particular, Par j 1.201 may be considered a shorter variant of Par j 1.0102 (5, 6).

The Par j 2 clone, isolated from a cDNA gene library, contains an insert of 622 nt with a correct reading phase of 133 amino acids and a signal peptide of 31 amino acids. The mature protein contains 102 amino acids and has a molecular weight of 11.344 Da (FIG. 1, panel C). At the amino acid level, it shows 45% homology with Par j 1 and is also a major allergen since it reacts with almost all sera of allergic subjects (7). Despite their structural homology, Par j 1 and Par j 2 are two independent allergens, as demonstrated by cross-reactivity experiments (7). Moreover, when a pool of sera from allergic subjects is preincubated with recombinant allergens Par j 1 and Par j 2, the IgE binding in the 10-14 kDa region of the Pj pollen is completely inhibited, suggesting that only these two allergens are present in this region and that together they are capable of inhibiting the majority of specific IgE against Pj allergens (7).

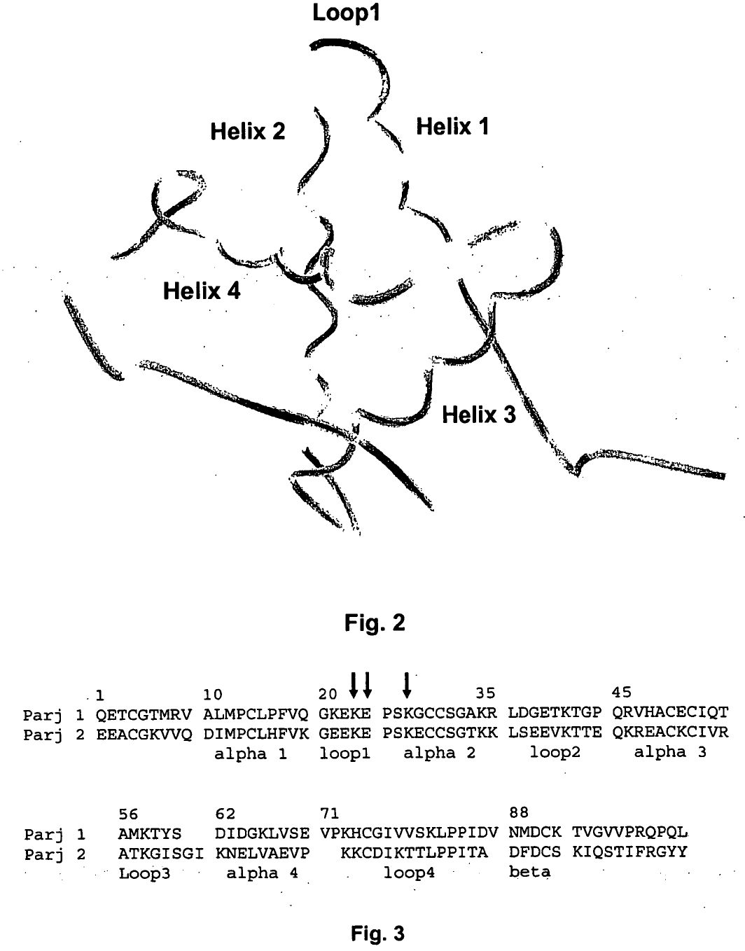

Furthermore, a search of the EMBL data bank revealed that Par j 1 and Par j 2 belong to a family of proteins known as nonspecific lipid transfer proteins (ns-LTP) that can transport lipid molecules across the cell membrane. These proteins share numerous characteristics such as the presence of eight cysteines that can form four disulphide bridges with a well-preserved α-α-α-α-β secondary structure (FIG. 2) (8). Since Par j 1 presents all the characteristics of ns-LTP, a structural model was built using the soybean ns-LTP crystal as reference. According to this model, Par j 1 and Par j 2 present four sulfide bridges in the order: 4-52, 14-29, 30-75, 50-91. Moreover, by applying a site-directed mutagenic strategy, we demonstrated the importance of the disulfide bridges in the formation of IgE epitopes and the existence of a dominant epitope in the loop1 region from amino acid 1 to 30 (FIG. 3 and [8]).

While allergy symptoms can be treated pharmacologically, the only preventive therapy is specific immunotherapy (SIT), wherein diluted quantities of the allergen are administered subcutaneously to suppress specific reactivity against the allergen (9). Most commercially available protein extracts are, however, crude extracts, a mixture of numerous allergenic components which are difficult to standardize.

Moreover, such a strategy may involve the administration of allergenic components to which the patient is not sensitive, thus inducing the production of specific IgE against other components in the extract (10). In addition, the administration of the allergen in toto carries the risk of side effects that may also cause anaphylactic shock. To eliminate these drawbacks, one of the principal objectives is to characterize and to develop alternative molecules with fewer side effects, i.e. molecules that do not interact with IgE. The modification of native allergens in an attempt to generate molecules with reduced risk of anaphylaxis has been proposed by March and co-workers, who suggested polymerizing crude extracts with formaldehyde or gluteraldehyde (11). Clinical trials demonstrated the efficacy of these modified molecules, but they still had all the drawbacks described above regarding the difficulty of accurately standardizing the extracts. However, the possibility to genetically modify allergens represents a more reliable solution because knowledge of the nucleotide sequence, and hence the amino acid sequence of such molecules, allows the production of proteins mutated in a pure form that is absolutely reproducible.

Patent application WO 02/20790 concerns the variants of the family of ns-LTP allergens, to which the allergens of the present invention belong, with a reduced ability to form disulfide bridges.

Patent application WO 02/22674 concerns that hypoallergenic variants of the major allergen Par j 2 in which lysine residues are substituted or deleted.

However, both documents of previous technology concern variants of a specific allergen and do not produce a molecule engineered to contain multimers of single allergens and/or regions deriving from different allergens that can be advantageously used as a single hypoallergenic principle.

Hence, the object of the present invention is a multimer protein molecule comprising at least a first amino acid sequence having substantially the sequence of one of the Parietaria judaica major allergens Par j 1 or Par j 2 and a second amino acid sequence having substantially the sequence of one of the Parietaria judaica major allergens Par j 1 or Par j 2.

In a preferred embodiment, the multimer protein molecule of the invention also contains at least a third sequence of one of the Parietaria judaica major allergens Par j 1 or Par j 2.

In a preferred embodiment, the sequence of the major allergen Par j 1 is substantially Seq Id No. 1 and the sequence of the major allergen Par j 2 is substantially the sequence Seq Id No. 3.

In a preferred embodiment, the sequence of the Parietaria judaica major allergen Par j 1 and/or of the major allergen Par j 2 is mutated in the amino acid region of loop 1, substantially comprised from amino acid 1 to 30 of Seq Id No. 1 and/or Seq Id No. 3. Preferably, the mutated sequence of the major allergen Par j 1 is substantially the sequence Seq Id No. 2; preferably the mutated sequence of the major allergen Par j 2 is substantially sequence Seq Id No. 4. More preferably, the mutated sequence of the major allergen Par j 1 is substantially sequence Seq Id No. 2 and the mutated sequence of the major allergen Par j 2 is substantially sequence Seq Id No. 4.

A further object of the invention is a nucleic acid that encodes the multimer protein molecule of the invention.

A further object of the invention is a recombinant vector for expression in prokaryotic cells, comprising, under the control of a suitable transcription promoter system, the nucleic acid of the invention.

A further object of the invention is a recombinant vector for expression in eukaryotic cells, comprising, under the control of a suitable transcription promoter system, the nucleic acid of the invention.

A further object of the invention is a multimer protein molecule according to the invention for medical use, preferably as a hypoallergenic.

A further object of the invention is a pharmaceutical composition comprising an effective and acceptable amount of the multimer protein molecule according to the invention and suitable adjuvants and/or diluents.

The invention is described below in non limiting examples with reference to the following figures:

FIG. 1. Amino acid sequences of major allergen Par j 1 (A) (Seq Id. No. 1) and Par j 2 (C) (Seq Id No. 3). Sequence B shows the hypoallergenic derivative of Par j 1 mutated in the loop 1 region (Seq Id No. 2); sequence D shows the hypoallergenic derivative of Par j 2 in the loop 1 region (Seq Id No. 4); underlined amino acids show inserted mutations.

FIG. 2. Schematic diagram of the three-dimensional model of Par j 1 illustrating the structure composed of four alpha helices typical of the ns-LTP family.

FIG. 3. Alignment of the amino acid sequences of Par j 1.0102 and Par j 2.0101 in relation to their three-dimensional structure. The numbers refer to the Par j 1.0102 sequence. Arrows indicate substituted amino acids.

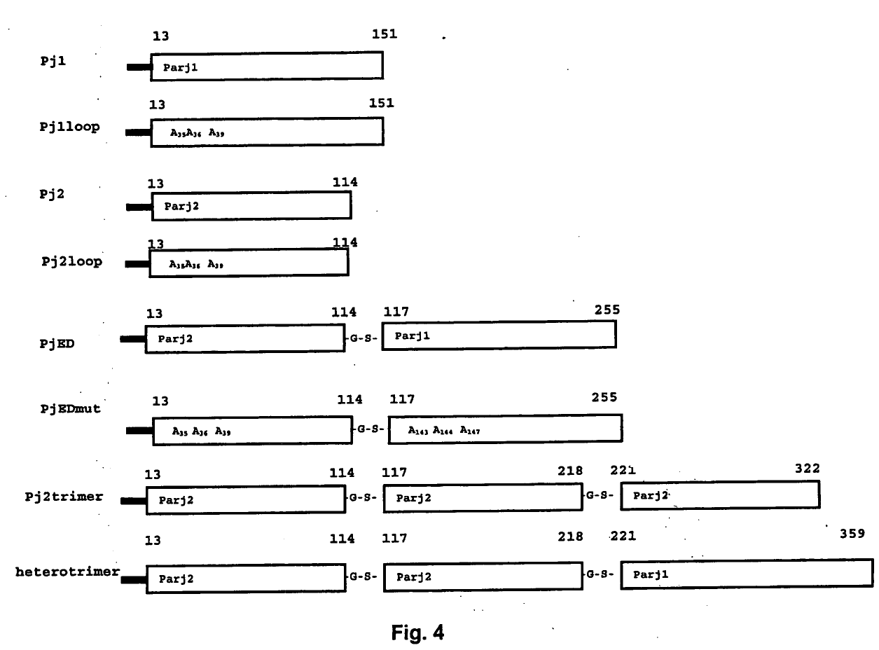

FIG. 4. Schematic diagram of plasmid constructs. The numbers indicate the position of the amino acids starting from the first methionine as expressed by the pQE30 and pQE31 expression vectors used for the expression of recombinant proteins. Added or substituted amino acids are indicated.

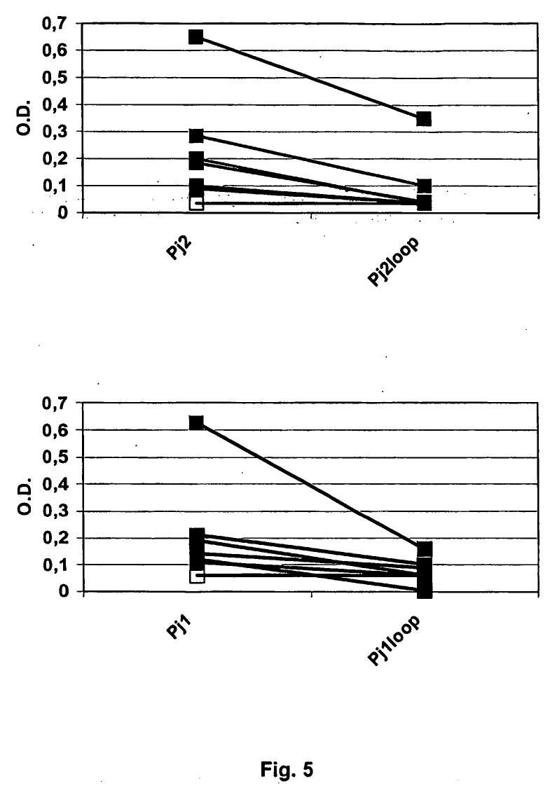

FIG. 5. ELISA test showing the ability of the Pj1 loop and Pj2 loop mutants to bind to the human IgE as compared to respective wild-type molecules. The lines with black squares denote sera of Pj-allergic subjects; the line with white squares denotes the binding activity of serum from a non-allergic subject.

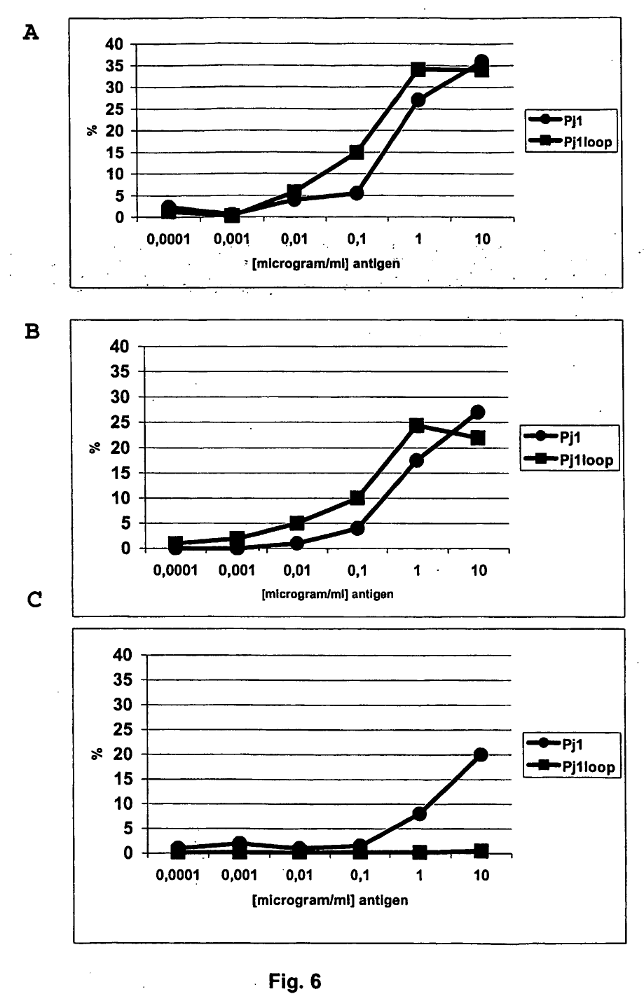

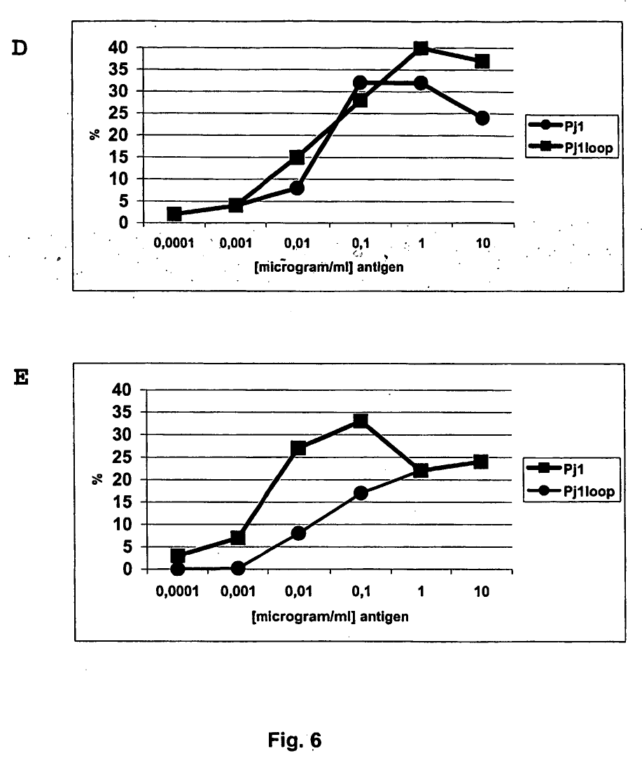

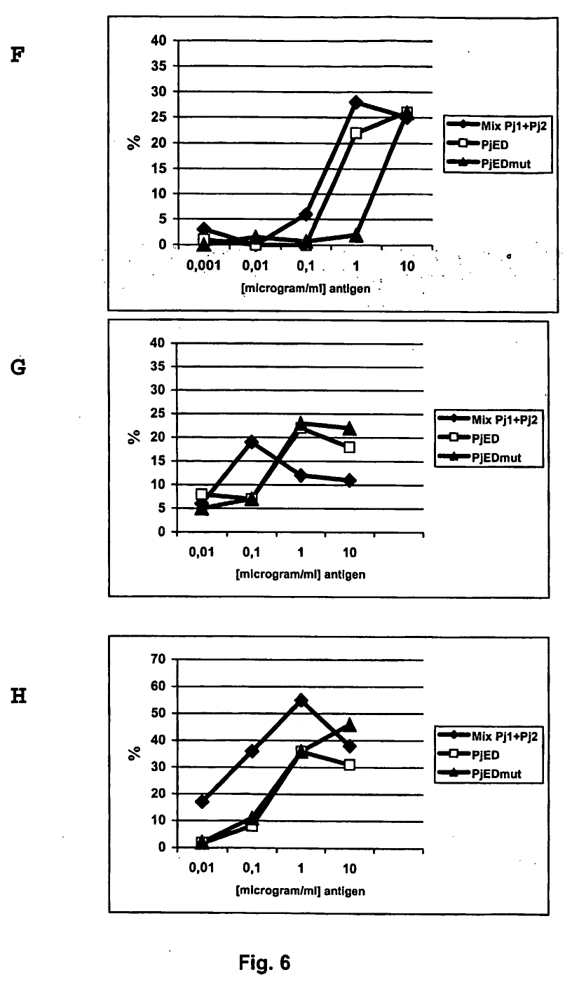

FIG. 6. Histamine release profiles of Pj1 loop, PjED and PjEDmut mutants. Panels A-E show the amount of histamine released by the Pj 1 loop mutant and the Par j 1 wild-type molecule. Panels F-H indicate the amount of histamine released by the PjED and PjEDmut mutants compared with a mix containing an equimolecular quantity of the Par j 1 and Par j 2 allergens. Each panel represents one allergic patient. The values on the x-axis indicate the concentration of antigen in μg/ml. The values on the y-axis express the percentage of released histamine compared with the total amount of histamine.

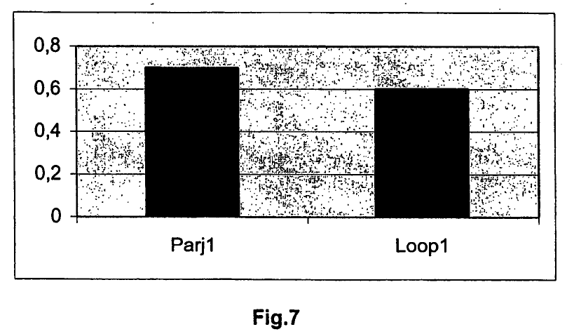

FIG. 7. ELISA test using the wild-type Par j 1 (Pj1) and Pj1 loop molecules as antigens. The values on the y-axis indicate the binding ability of the molecules compared with a polyclonal antibody against the Par j 1 molecule.

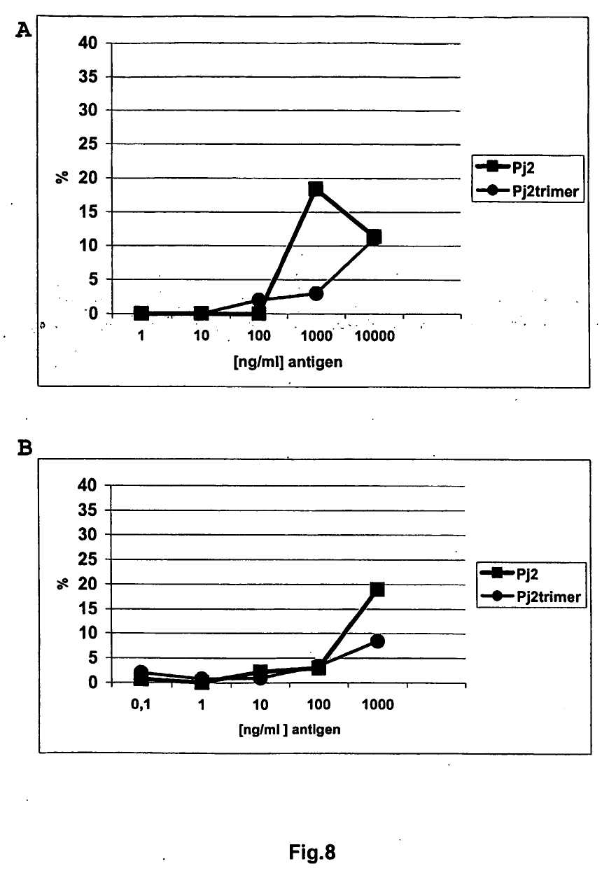

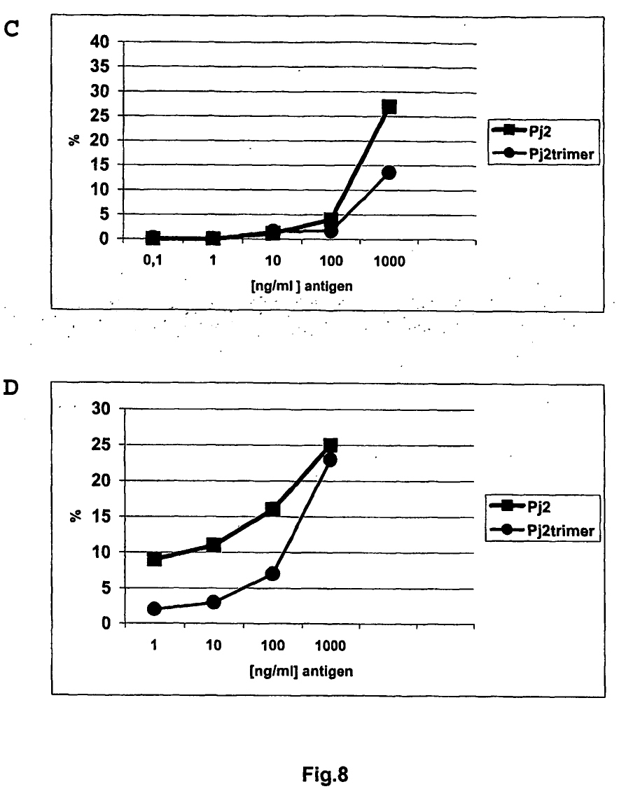

FIG. 8. Histamine release assay of the Pj2 trimer mutant compared with an equimolecular amount of the monomer Par j 2 allergen. Panels A-D indicate the amounts of histamine released by each allergic patient. The x-axis indicates the antigen concentration expressed in ng/ml. The y-axis expresses the percentage of released histamine compared with the total amount of histamine.

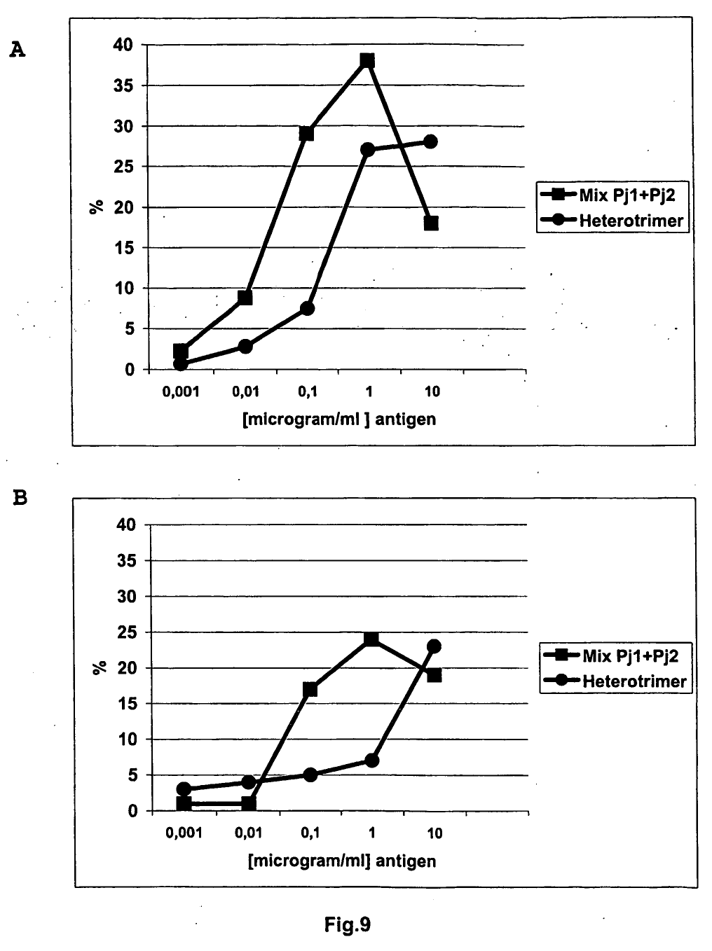

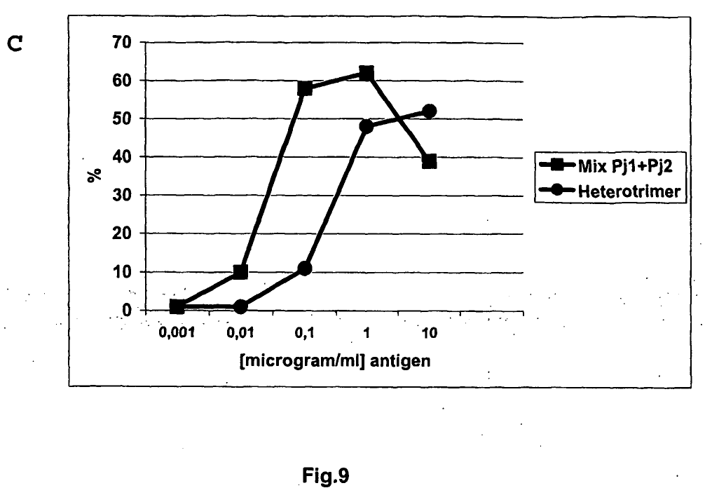

FIG. 9. Histamine release profile of the heterotrimer mutant compared with Par j 1 and Par j 2 monomers. Panels A-C indicate the quantity of histamine released by each patient. The values on the x-axis indicate the antigen concentration expressed in μg/ml. The values on the y-axis express the percentage of histamine released compared with the total amount of histamine.

### MATERIALS AND METHODS

Cloning and Expression of Wild-Type Par j 1 and Par j 2

To produce the Pj major allergen Par j 1, the pQE30 prokaryote vector (Qiagen) was used. The vector is able to express recombinant proteins fused to a short histidine tail, in an inducible way by means of isopropyl-β-D-thiogalactoside (IPTG). The histidine residues permit the purification of the recombinant protein by affinity chromatography. The P5 clone containing the processed version of Par j 1 (EMBL accession number X77414) underwent one cycle of DNA polymerase chain reaction (PCR) under the conditions: 94° C. for 1 min, 52° C. for 1 min, 72° C. for 1 min for 30 cycles. The synthetic oligonucleotides used were:

Par j 1.0102 forward

(Seq Id No. 5)

(5′ ATT GGATCCCAAGAAACCTGCGGGACTATG 3′)
Par j 1.0102 reverse

(Seq Id No. 6)

(5′ ATTAAGCTTGGCTTTTTCCGGTGCGGG 3′)

(bold letters denote the restriction enzyme sequences used in the process). The major allergen Par j 2 was generated using the same vector and the same methods described for Par j 1, except for the oligonucleotides and the template. To this end, the templates for sequencing the P2 clone (EMBL accession number X95865) and the oligonucleotides were:

Par j 2 forward

(Seq Id No. 7)

(5′ CCTGGATCCGAGGAGGCTTGCGGG 3′)
and
Par j 2 reverse

(Seq Id No. 8)

(5′ GCGAAGCTTATAGTAACCTCTGAAAATAGT 3′)

(bold letters denote the restriction enzyme sequences used in the process).

The generated fragments were then fractionated on 1% agarose gel in 1× TBE, extracted from the gel, purified and digested with the Bam HI and Hind III restriction enzymes. The pQE30 vector was digested using the same restriction enzymes. The linearized vector and the digested fragment were incubated at 16° C. for 4 hours in the presence of DNA ligase enzyme according to various stoichiometric ratios. The reaction mixture was used to transform the bacterial strain M15. The recombinant clones were isolated and the plasmid DNA was sequenced using the Sanger method. The resulting nucleotide sequence showed that the DNA fragment inserted in the pQE30 vector was identical to our previously published results.

Construction of Par j 1 and Par j 2 Loop1 Mutants

The point mutants of Par j 1 and Par j 2 were generated by PCR using as template the cDNA described above that encodes the wild-type versions of these allergens. In particular, for the Pj 1 loop clone the following oligonucleotides were used: Par j 1-fwd (5′-ATTGGATCCCAAGAAACCTGCGGGACTATG-3′) (Seq Id No 9) LOOP1 mut-rev (5′-AAACTGCAGCACCCCgcTGACGGCgCTgcCTCTTTCC-3′) (Seq Id No. 10), to synthesize a DNA fragment encoding the first 30 amino acid terminals of Par j 1, in which the amino acids Lys23, Glu24, Lys27 were substituted with a neutral alanine amino acid. The generated DNA fragment was then digested at 5′ and 3′ with the restriction enzymes Bam HI and Pst I, respectively, and in frame introduced in the Pj1 expression vector digested with the same restriction enzymes (bold letters denote the restriction enzymes sites used for cloning; lower case letters denote the substituted nucleotides for mutagenesis; see FIG. 1, panel B and FIG. 3, FIG. 4 for numbers and position of the amino acids).

The following nucleotides were used for the Pj2 loop clone:

Par j 2 fwd

(Seq Id No 11)

(5′-GTGGGATCCGAGGAGGCTTGCGGGAAAGTGGTGCAG-3′)
Par j 2 mut-rev

(Seq Id No 12)

(5′-AAACTGCAGCACTCCgcCGACGGCgCCgcCTCCTCCC-3′),

to synthesize a DNA fragment encoding the first 30 amino acids of the Par j 2amino acid terminal in which the amino acids Lys23, Glu24, Lys27 were substituted with the neutral alanine amino acid. The generated DNA fragment was then digested at 5′ and 3′ with the restriction enzymes Bam HI and Pst I, respectively, and introduced into the Pj2 expression vector digested with the same restriction enzymes (bold letters denote the restriction enzyme sites used for cloning; lower case letters denote the substituted nucleotides for mutagenesis; see FIG. 1, panel E and FIG. 3, FIG. 4 for numbers and position of the amino acids).
Construction of a Dimer Molecule Containing the Genetic Information for Wild-Type Par j 1 and Par j 2 and the Mutatedform in the Loop1 Region

To construct a heterodimer molecule containing the genetic information for the major allergens Par j 1 and Par j 2, a DNA fragment was initially generated that contained the wild-type Par j 2 sequence using the synthetic oligonucleotides:

Pj2-for

(Seq Id No. 13)

(5′-GTGGGATCCGAGGAGGCTTGCGGGAAAGTGGTGCAG-3′),
and
Pj2-rev

(Seq Id No. 14)

(5′-CGCGGATCCATAGTAACCTCTGAAAATAGT-3′)

the Pj2 clone as a template and under the same PCR conditions: 94° C. for 1 min, 52° C. for 1 min, 72° C. for 1 min for 30 cycles. The resulting fragment was then purified and digested with the Bam HI restriction enzyme and inserted in the Pj1 plasmid previously digested with the same restriction enzyme. The recombinant plasmids containing a copy of the Par j 2 fragment inserted in the correct orientation were isolated and assayed for their ability to express stable recombinant multimer proteins (dimers Parj2-Parj1) (FIG. 4, PjED clone). By introducing the restriction sites used for cloning the fragment, two amino acids (G and S) were introduced at the level of the junction sites, without shifting the correct reading phase.

The heterodimer containing a copy of each of the Par j 1 and Par j 1 allergens mutagenized in the loop1 region was generated using a PCR strategy identical to that described for the formation of the Parj1-Parj2 heterodimer, modifying only the template used for PCR. In particular, the Pj2 loop clone was amplified using the Pj2-for and Pj2-rev oligonucleotides. The resulting fragment was then purified and digested with the restriction enzyme Barn H1 and inserted in the Pj1 loop plasmid previously digested with the same restriction enzyme. The recombinant plasmids containing a copy of the Pj2 loop fragment inserted in the correct orientation were isolated and assayed for their ability to express stable recombinant dimers (Parj2-Parj1 dimers both mutated in the loop1 region) (FIG. 3 and FIG. 4 [PjEDmut clone]). By introducing the restriction sites for cloning the fragments, two amino acids (G and S) were introduced at the level of the junction sites that do not modify the correct reading phase (FIG. 4 heterodimer).

Construction of a Multimer Par j 2 Molecule and a Heterodimer Containing both Major Allergens.

To construct the Par j 2 trimer, the plasmid vector pQE3 1, the DNA encoding the Par j 2 recombinant protein and the XLI blue bacterial strain of E. coli were used. The four restriction endonucleases Barn HI, Sac I Sal I and Hind III were used.

To insert the restriction enzyme sites at the boundaries of the Parj2 DNA sequence without altering the sequence itself, the Par j 2 DNA was amplified by PCR using the following primers:

| | |
|---|---|
| Direct Bam HI: | |
| 5′- CCTGGATCCTGAGGAGGCTTGCGGG-3′ | (Seq Id No. 15) |
| Reverse Sac I: | |
| 3′- CCTGAGCTCATAGTAACCTCTGAA-5′ | (Seq Id No. 16) |
| Direct Sac I: | |
| 5′- CCTGAGCTCGAGGAGGCTTGCGGG-3′ | (Seq Id No. 17) |
| Reverse Sal I: | |
| 3′- CCTGTCGACATAGTAACCTCTGAA-5′ | (Seq Id No. 18) |
| Direct Sal I: | |
| 5′- CCTGTCGACGAGGAGGCTTGCGGG-3′ | (Seq Id No. 19) |
| Reverse Hind III: | |
| 3′- CCTAAGCTTCTAATAGTAACCTCT-5′ | (Seq Id No. 20) |

and under the following conditions: 94° C. for 1 min, 52° C. for 1 min, 72° C. for 1 min for 30 cycles (bold letters denote the restriction enzyme sites).

To construct the recombinant plasmid containing the first Par j 2 monomer, CDNA containing the genetic information for the Par j 2 antigen was amplified using the Direct Bam HI and Reverse Sac I primers. In this way, the PCR products were digested without disrupting the nucleotide sequence encoding the Par j 2 protein. The digested DNA was then bound in the correct reading phase to the pQE31 vector digested with the same enzymes.

The resulting recombinant plasmid was then used to transform the bacterial strain XL1blue; the positive clones induced with IPTG were assayed by Western blot analysis and hybridization with the His-probe that recognizes the six histidine residues present in the recombinant protein. The correctness of cloning was confirmed by sequencing the recombinant plasmid DNA.

To construct the dimer clone, the plasmid construction was digested with Sac I and Sal I enzymes. The linearized plasmid was then incubated with the DNA fragment containing the Par j 2 allergen after amplification by PCR using the direct Sac I and reverse Sal I primers. The recombinant clones were analyzed and controlled as described previously. To construct the clone containing the trimer construct, the plasmid DNA containing the Par j 2 dimer was digested with Sal I and Hind m enzymes, and then incubated in a ligase reaction, with a DNA fragment containing the information for the Par j 2 amplified with the direct Sal I and reverse Hind III primers. It should be noted that in this clone a stop codon is inserted. Cloning is confirmed by sequencing the recombinant plasmid DNA.

By introducing restriction sites to clone the fragments, two amino acids (G and S) were introduced at the level of the junction sites that do not modify the correct reading frame (FIG. 4 heterotrimer).

To construct heteromultimer molecules containing both major allergens Parj1 and Parj2, the following strategy described previously for the PjED clone was used. Recombinant plasmids containing two copies of the Par j 2 fragment inserted in the correct orientation were isolated and assayed for their ability to express stable recombinant multimer proteins (Parj2-Parj2-Parj1 trimers). By introducing restriction sites used for cloning the fragment, two amino acids (G and S) were introduced at the level of the junction sites that do not modify the correct reading frame (FIG. 4, heterotrimer).

Induction and Purification of Recombinant Proteins

Ten ml of an o/n culture were used to inoculate a 400 ml of 2YT culture medium containing ampicillin and kanamycin at a final concentration of 100 μg/ml and 10 μg/ml, respectively. The culture was grown at 37° C. under agitation. After 2 hours, IPTG is added to the culture at a final concentration of 1 mM, and the culture is grown for 4 hours at 37° C. under agitation. At the end of this step, the bacterial culture is centrifuged at 5000 rpm for 15 minutes. The pellets are suspended in Start buffer (10 mM Na phosphate pH 7.4 and 6 M urea) and cells are sonicated. The lysate is then centrifuged at 14.000 rpm for 30 min, and the clarified lysate is loaded on a CM Sepharose (Pharmacia) column. The proteins are diluted using a discontinuous gradient of NaCl, and the fractions containing the protein of interest are dialyzed for 2 hours against a buffer containing 10 mM Na phosphate pH 7.4 and 1 M NaCl to allow the formation of the native three-dimensional structure. The recombinant proteins were then definitively purified using a His Trap column (Amersham) following the manufacturer's instructions. The diluted fractions were then analyzed on 16% polyacrylamid gel, and the fractions containing the recombinant protein were quantitatively evaluated by spectrophotometry after staining by the Bradford method. Lastly, the proteins were desalted using a Sephadex G-25 column (Pharmacia).

The proteins produced by recombination technique were electrophorated on 16% SDS polyacrylamid gel. Their purity and concentration were determined by Coomassie Brilliant Blue staining.

ELISA

The ELISA test was carried out as described in Bonura et al. (13). The concentration of the antigen used in each well was 5 μg/ml. The patients (n=8) had a clear history of allergy to Parietaria judaica and all tested positive to the skin test using commercially available products.

Histamine Release Assay

The histamine release assay was performed using heparinized blood drawn from Pj-allergic subjects and using a concentration scale of allergen from 0.0001 μg/ml to 10 μg/ml. The release protocol was carried out as described previously (13). S-adenosyl-L-methionine-H³ (Amersham, UK) was used as a radioactive reaction marker for the presence of the methyltransferase histamine enzyme prepared from male rat kidney. The total quantity of histamine was calculated by measuring the radioactivity of 100 μg/ml of blood diluted with 1 volume of 0.05 M phosphate buffer ph 7.9, and after boiling the sample for 10 minutes. The spontaneous release was calculated by incubating the sample in the presence of the dilution buffer. The percentage of released histamine was calculated as the percentage of histamine released after subtracting the percentage spontaneously released by the sample without stimulation.

### RESULTS

Allergy to Parietaria judaica pollen is one of the most common forms of allergy in the Mediterranean area. In particular, Par j 1 and Par j 2 are the two major players in allergic reaction and therefore represent two chief targets in the search for products to be used in specific immunotherapy for treating Pj allergy. These two independent allergens present similar characteristics in that they belong to the ns-LTP family. This family of plant proteins has been characterized at the structural level. All its components possess a highly compact structure comprising four alpha helices (see FIG. 2) brought together by eight cysteine residues that form four sulfur bridges in the order 4-52, 14-29, 30-75, 50-91 (the numbers refer to the primary sequence of mature Par j 1 shown in FIG. 1, panel A). All strategies described below aim to create molecules with a reduced allergenic capability, while maintaining a three-dimensional structure similar to the native counterparts. In particular, the site-directed mutagenesis of amino acids Lys23, Glu24, Lys27 of Par j 1 and Par j 2 comprised in the region defined loop1 (see FIG. 1, FIG. 2, FIG. 3) have shown they can enormously influence the binding ability with human IgE. In FIG. 5, an ELISA analysis demonstrates a sharp reduction in IgE binding for Pj 1 loop and Pj 2 loop mutants, reaching a level of activity comparable with that of the serum of a non-allergic patient. FIG. 6 shows the histamine release profile when the whole blood of Pj-allergic patients (n=5) is incubated with increasing quantities of the Pj1 loop or the wild-type mutant. In 40% of these patients the percentage of release was lower than that released by the wild-type molecule (FIG. 6, panels C and E). The remaining patients showed a certain heterogeneity of response, with profiles comparable to that of the wild-type molecule. This type of mutagenesis does not seem to interfere with the general structure of the protein. FIG. 7 shows an ELISA test that compared the wild-type Pj and the Pj1 loop in relation to their ability to bind a polyclonal rabbit antibody obtained by immunization with the Pj1 wild-type molecule. The analysis shows a high percentage of binding also for the Pj1 loop molecule, demonstrating the persistence of numerous protein structure domains comparable to those of the original molecule.

This strategy shows that modification of only three amino acids, located in a region exposed to a solvent that does not interfere with the three-dimensional structure of the protein (see model described in FIG. 2 and data in FIG. 7), can considerably reduce the quantity of specifically bound IgE compared with the respective wild-type counterparts (see FIG. 6). This suggests that the molecules are a valid solution and are applicable to the entire population of Pj-allergic subjects. In light of these data and based on the observation that both allergens are able to induce the production of IgE in Pj-allergic subjects, it was decided to generate a new class of molecules formed by the head-to-tail linking of two single allergens in the attempt to generate a single pharmacological formulation to be used in Pj pollen desensitization therapy. Furthermore, for other allergenic sources it was shown that the multimerization of single peptides can result in the formation of proteins with reduced anaphylaxis (11, 12). To this end, a plasmid construction composed of the head-to-tail linking of Par j 2 and Par j 1 was generated in their native versions (for details see FIG. 4 PjED clone), and, simultaneously, a plasmid construction composed of the head-to-tail linking of Pj1 loop and Pj2 loop clones (FIG. 4, PjEDmut clone). The mutant contains the sequences encoding the two major allergens both modified in the amino acids in positions 23, 24 and 27 (FIG. 1). The binding activity of the PjED and PjEDmut clones was assayed using the histamine release technique. Their activity was compared with that of an equimolecular mix of the two Par j 1 and Par j 2 monomers. An analysis of the graphs in FIG. 6, panels F, G and H shows that the association of the two allergens (PjED clone) results in a lower ability of histamine release in certain subjects (FIG. 6, panels G and H), and more markedly, the double mutant (PjEDmut) has a reduced ability to release histamine in one of the patients studied (FIG. 6, panel F).

In light of these results for the PjEd and PjEDmut mutants, where an event of polymerization in itself can in some patients reduce anaphylaxis, it was decided to generate multimers in which various pairs of the Par j 1 and Par j 2 were fused. In detail, the Pj2 trimer mutant is composed of three pairs of head-to-tail linked Par j 2 allergen copies, as shown in FIG. 4. The molecule was studied in a histamine release test on four Pj-allergic subjects. All subjects presented a marked reduction in histamine release in that their activity was comparable to that of monomer Par j 2 (FIG. 8). Similarly, when two pairs of the Par j 2 allergen were linked head-to-tail with a pair of the Par j 1 allergen (FIG. 4, heterotrimer clone), a hybrid multimer molecule is obtained in which the allergenic characteristics were studied by comparing them to the activity of their monomer allergen counterparts. Also in this case, a reduced release of histamine was observed in the blood of the allergic patients (FIG. 9).

In conclusion, the design, production and allergy analysis is described of two families of mutants of two Pj major allergens, Par j 1 and Par j 2, with reduced allergenic activity.

### REFERENCES

[1] D'Amato, G. et al. (1998) Allergy 53, 567-78.

[2] Geraci, D., Oreste, U. and Ruffilli, A. (1978) Immunochemistry 15, 491-8.

[3] Corbi, A. L. and Carreira, J. (1984) Int Arch Allergy Appl Immunol 74, 318-23.

[4] Colombo, P., Duro, G., Costa, M. A., Izzo, V., Mirisola, M., Locorotondo, G., Cocchiara, R. and Geraci, D. (1998) Allergy 53, 917-21.

[5] Costa, M. A. et al. (1994) FEBS Lett 341, 182-6.

[6] Duro, G. et al. (1997) Int Arch Allergy Immunol 112, 348-55.

[7] Duro, G. et al. (1996) FEBS Lett 399, 295-8.

[8] Colombo, P. et al. (1998) J Immunol 160, 2780-5.

[9] Bousquet, J., Lockey, R. and Malling, H. J. (1998) J Allergy Clin Immunol 102, 558-62.

[10] Moverare, R., Elfinan, L., Vesterinen, E., Metso, T. and Haahtela, T. (2002) Allergy 57, 423-30.

[11] Marsh, D. G., Lichtenstein, L. M. and Campbell, D. H. (1970) Immunology 18, 705-22.

[12] Vrtala, S. et al. (2001) Faseb J 15, 2045-7.

[13] Bonura, A. et al. (2001) Int. Arch. Allergy Inmunol. 126, 32-40

1. A multimer protein molecule comprising at least a first amino acid sequence having substantially the sequence of one of the Parietaria judaica major allergens Par j 1 or Par j 2 and a second amino acid sequence having substantially the. sequence of one of the Parietária judaica major allergens Par j 1 or Par j 2.

2. The multimer protein molecule according to claim 1 further comprising at least a third sequence of one of the Parietaria judaica major allergens Par j 1 or Par j 2.

3. The multimer protein molecule according to claim 1, wherein the sequence of the major allergen Par j 1 is substantially the sequence Seq Id No. 1 and the sequence of the major allergen Par j 2 is substantially the sequence Seq Id No. 3.

4. The multimer protein molecule according to claim 1, wherein the sequence of the Parietaria judaica major allergen Par j 1 and/or of the major allergen Par j 2 is mutated in the loop 1 amino acid region, substantially comprising from amino acid 1 to 30 of Seq Id No. 1 and/or of Seq Id No. 3.

5. The multimer protein molecule according to claim 4, wherein the mutated sequence of the major allergen Par j 1 is substantially the sequence Seq Id No. 2.

6. The multimer protein molecule according to claim 4, wherein the mutated sequence of the major allergen Par j 2 is substantially the sequence Seq Id No. 4.

7. The multimer protein molecule according to claim 4, wherein the mutated sequence of the major allergen Par j 1 is. substantially the sequence Seq Id No. 2 and the mutated sequence of the major allergen Par j 2 is substantially the sequence Seq Id No. 4.

8. A nucleic acid that encodes the multimer protein molecule according to claim 1.

9. A recombinant vector for expression in prokaryotic cells comprising, under the control of a suitable transcription promoter system, the nucleic acid according to claim 8.

10. A recombinant vector for expression in eukaryotic cells comprising, under the control of a suitable transcription promoter system, the nucleic acid according to claim 8.

11. The multimer protein molecule according to claim 1 for medical use.

12. The multimer protein molecule according to claims 1 for medical use as a hypoallergenic agent.

13. A pharmaceutical composition comprising an effective and acceptable amount of the multimer protein molecule according of claims 1 and suitable adjuvants and/or diluents.


SEQUENCE LISTING (20 sequences)

SEQ ID NO: 1 - protein, 139 aa, Parietaria judaica
QETCGTMVRALMPCLPFVQGKEKEPSKGCCSGAKRLDGETKTGPQRVHACECIQTAMKTYSDIDGKLVSEVPKHCGIVDSKLPPIDVNMDCKTVGVVPRQPQLPVSLRHGPVTGPSDPAHKARLERPQIRVPPPAPEKA

SEQ ID NO: 2 - protein, 139 aa, Artificial Sequence
  mutagenized Parj1
QETCGTMVRALMPCLPFVQGKEAAPSAGCCSGAKRLDGETKTGPQRVHACECIQTAMKTYSDIDGKLVSEVPKHCGIVDSKLPPIDVNMDCKTVGVVPRQPQLPVSLRHGPVTGPSDPAHKARLERPQIRVPPPAPEKA

SEQ ID NO: 3 - protein, 102 aa, Parietaria judaica
EEACGKVVQDIMPCLHFVKGEEKEPSKECCSGTKKLSEEVKTTEQKREACKCIVRATKGISGIKNELVAEVPKKCDIKTTLPPITADFDCSKIQSTIFRGYY

SEQ ID NO: 4 - protein, 102 aa, Artificial Sequence
  mutagenized Parj2
EEACGKVVQDIMPCLHFVKGEEAAPSAECCSGTKKLSEEVKTTEQKREACKCIVRATKGISGIKNELVAEVPKKCDIKTTLPPITADFDCSKIQSTIFRGYY

SEQ ID NO: 5 - DNA, 30 bp, Artificial Sequence
  primer
attggatcccaagaaacctgcgggactatg

SEQ ID NO: 6 - DNA, 27 bp, Artificial Sequence
  primer
attaagcttggctttttccggtgcggg

SEQ ID NO: 7 - DNA, 24 bp, Artificial Sequence
  primer
cctggatccgaggaggcttgcggg

SEQ ID NO: 8 - DNA, 30 bp, Artificial Sequence
  primer
gcgaagcttatagtaacctctgaaaatagt

SEQ ID NO: 9 - DNA, 30 bp, Artificial Sequence; identical to SEQ ID NO: 5
  primer

SEQ ID NO: 10 - DNA, 37 bp, Artificial Sequence
  primer
aaactgcagcaccccgctgacggcgctgcctctttcc

SEQ ID NO: 11 - DNA, 36 bp, Artificial Sequence
  primer
gtgggatccgaggaggcttgcgggaaagtggtgcag

SEQ ID NO: 12 - DNA, 37 bp, Artificial Sequence
  primer
aaactgcagcactccgccgacggcgccgcctcctccc

SEQ ID NO: 13 - DNA, 36 bp, Artificial Sequence; identical to SEQ ID NO: 11
  primer

SEQ ID NO: 14 - DNA, 30 bp, Artificial Sequence
  primer
cgcggatccatagtaacctctgaaaatagt

SEQ ID NO: 15 - DNA, 25 bp, Artificial Sequence
  primer
cctggatcctgaggaggcttgcggg

SEQ ID NO: 16 - DNA, 24 bp, Artificial Sequence
  primer
cctgagctcatagtaacctctgaa

SEQ ID NO: 17 - DNA, 24 bp, Artificial Sequence
  primer
cctgagctcgaggaggcttgcggg

SEQ ID NO: 18 - DNA, 24 bp, Artificial Sequence
  primer
cctgtcgacatagtaacctctgaa

SEQ ID NO: 19 - DNA, 24 bp, Artificial Sequence
  primer
cctgtcgacgaggaggcttgcggg

SEQ ID NO: 20 - DNA, 24 bp, Artificial Sequence
  primer
cctaagcttctaatagtaacctct